(19) United States
(12) Patent Application Publication
(10) Pub. No.: US 2014/0114128 A1
(43) Pub. Date: Apr. 24, 2014

(54) APPLICATOR FOR AN ENDOSCOPE

(71) Applicant: Anthony Arthur Wills, London (GB)

(72) Inventor: Anthony Arthur Wills, London (GB)

(73) Assignee: ENDOGUARD LIMITED, Bury St. Edmunds (GB)

(21) Appl. No.: 14/008,844

(22) PCT Filed: Mar. 30, 2012
(86) PCT No.: PCT/GB2012/000299

§ 371 Date: Nov. 22, 2013

(30) Foreign Application Priority Data
Mar. 30, 2011 (GB) 1105401.2

(51) Int. Cl. A61B 1/00; A61F 7/00; A61F 7/12; A61B 17/00

(52) U.S. Cl. CPC A61B 1/00154; A61B 17/00234; A61F 7/007; A61F 7/12
USPC 600/114

(57) ABSTRACT

A device for applying an attachment to an endoscope is disclosed. The device comprises an applicator supporting the attachment, an end of the attachment being accessible such that the endoscope is insertable into the attachment in a longitudinal direction. The attachment is removably secured to the applicator to enable the attachment to be withdrawn from the applicator together with the endoscope.

The present invention relates to a device for applying an attachment to an endoscope.

Endoscopes are used in minimally invasive surgery (MIS) by surgeons to permit remote visualisation and navigation within a body cavity inside a patient. They act as the eyes of the surgeon whilst a surgical procedure, tissue manipulation or diagnostic investigation is undertaken. One type of endoscope is a laparoscope for abdominal MIS, which is used in speciality areas such as a laparoscopic general surgery including upper and lower gastrointestinal surgery, gynaecology, obesity surgery (bariatric surgery) and urology, as well as other surgical sectors utilising a rigid scope or semi rigid scope, such as thoracic and pulmonary, ENT, and neurological surgery.

Minimally invasive surgery (MIS), often referred to as “keyhole surgery”, as well as Minimum Access Surgery (MAS), is defined as a surgical method using small abdominal skin incisions (or no abdominal skin incisions, in which case a natural orifice is used in conjunction with an internal incision), as opposed to classic open surgical procedures that require large incisions. In MIS, a special access port called a cannula (which has a pre-determined internal diameter to allow instrument access and inter-change) is inserted into the skin incision. A miniature camera is introduced into the body through the cannula and transmits images to a video monitor, thereby allowing a physician to visualise, diagnose and, if necessary, treat a variety of conditions.

MIS is already an integrated part of daily surgical activity in surgical centres around the world. Many procedures are now performed by this “keyhole” approach using an appropriate endoscope, or by reduced open surgery (such as a mini-open or laparoscopically assisted procedures or hand assisted laparoscopic surgery or single incision laparoscopic surgery), where the skin incision is reduced compared with only a few years ago. The development of these MIS approaches is rapidly on-going and development of new techniques that will aid patients and society because of reduced complications, patient morbidity and hospital stay compared with the corresponding “old” methods will continue to drive the majority of procedures to MIS.

During a laparoscopic procedure, there are four main requirements for a surgeon or practitioner: continuous operative vision, maintained operative control, safety and time efficiency. The laparoscopic or endoscope lens in an MIS procedure is the surgeon's “eyes” and the optics regularly become soiled by peritoneum or other bodily fluid, blood, aerosol fat, tissue particulate, smoke, debris or condensation, all of which impair the surgeon's vision (via an external monitor/screen). These various soiling components are disturbed by various instruments introduced into the abdominal cavity via working ports, such as electro-cautery coagulation devices, laparoscopic scissors, ultrasonic coagulation cutting devices, suction-irritation devices and many others. Since these instruments are a crucial part of MIS and laparoscopic procedures, in general, they will remain as an adjunct to a source of lens contamination. As a result of this contamination, visualisation via the laparoscope optics is regularly diminished and impaired.

Prior art devices are known in which an attachment or sheath is applied to an endoscope to enable a channel to reach the distal end of the endoscope in order to provide access for instrumentation or to enable gas and/or liquid to flow across a lens surface at the distal end of the endoscope. These devices are generally applied manually, i.e. by hand, by a nurse in advance of the surgical procedure. There can be difficulties involved in applying the device to the endoscope, both in terms of ensuring that the device has been applied properly and is positioned correctly, as well as the risk of the device becoming damaged during the application process. These disadvantages often cause the devices to function inadequately and to be prone to breakage, thereby rendering them unsuitable for use in surgical procedures. In order for prior art devices to be of a sufficient strength to reduce the likelihood of breakage, the device has to be constructed out of thick material, which therefore increases the cross-sectional area taken up by the device and the endoscope, and therefore requires a larger cannula in order for the device to be used.

More recently, the present applicant has been making substantial progress in solving the above problems. Aspects of these developments have been detailed in two previously filed applications. International patent application publication No. WO 2011/004153 A1 discloses an accessory for partially enclosing a shaft of an endoscope. The accessory defines a first space for accommodating the shaft and a second space for defining a conduit along the shaft. In some embodiments, the conduit is for transporting fluid to and/or from the distal end of the endoscope. European Patent Application No. 10187123.4 discloses a flow guide for directing a fluid flow across an end surface of an endoscope in a controlled manner. In some embodiments, the flow guide is for guiding and directing a flow of gas to clear an end surface of the endoscope, in particular to clear a lens surface of the endoscope. Embodiments of the present invention provide applicator for an accessory and/or a flow guide as described in these applications, and the content of these two applications is incorporated by reference herein.

In a first aspect of the invention, there is provided a device for applying an attachment to an endoscope. The device comprises an applicator supporting the attachment. An end of the attachment is accessible such that the endoscope is insertable into the attachment in a longitudinal direction. The attachment is removably secured to the applicator to enable the attachment to be withdrawn from the applicator together with the endoscope.

Advantageously, the applicator enables the attachment to be applied to the endoscope more easily, and furthermore the likelihood of the attachment being damaged during the application process is significantly reduced. Rather than the manual application required for sheaths etc. in the prior art, application of the attachment to the endoscope is achievable by the user-friendly procedure of inserting the endoscope into the attachment and then subsequently withdrawing the endoscope from the applicator together with the attachment. This straightforward procedure may be easily carried out by anyone without training or specialist skill on the part of the user. The presence of the applicator also allows the attachment to be applied by the user without the user having to hold the attachment in his or her hand. A force applied by the user's hand to the attachment could be enough to damage the attachment, particularly if the attachment is made of a thin, delicate material. The applicator supports the attachment and therefore provides stability to it and hinders relative movement between the attachment and the applicator, thus making the insertion procedure easier and more reliable.

In some embodiments, the applicator provides a housing for the attachment and the attachment is supported in the housing and may be circumferentially surrounded by it. The location of the attachment within the housing protects the attachment from damage during transportation and storage of the device, as well as during application to the endoscope.

In some embodiments, the attachment comprises a stop having a surface for contacting the endoscope to define a predetermined maximum insertion distance of the endoscope into the attachment. The stop enables the exact placement of the attachment relative to the endoscope in the longitudinal direction. The stop makes the application of the attachment to the endoscope easier by ensuring that the user of the device will know that the attachment has been applied properly when the endoscope abuts the stop. This ensures that the device can be relied upon to reliably apply the attachment to the endoscope. In embodiments in which the attachment is used to provide a conduit for fluid to be applied to the distal end of the endoscope, it is important that the distal end of the endoscope is positioned correctly with respect to the attachment to enable fluid flow from the conduit to be directed optimally across the distal end of the endoscope.

In some embodiments, the surface is arranged to abut a portion of a distal end of the endoscope. This ensures that the longitudinal alignment of the attachment and the endoscope is defined directly at the distal end of the endoscope. This facilitates accurate alignment of the distal end of the endoscope, which in many cases, in particular for lens clearing, is the part of the endoscope at which alignment is most crucial.

In some embodiments, the applicator comprises a wall adjacent the stop for exerting a reaction force to a longitudinal force applied to the surface. The wall protects the stop from damage as the endoscope is inserted into the attachment. Without the presence of the wall, a sufficient force applied by the user inserting the endoscope in the longitudinal direction could cause the stop to buckle, and/or break off from the attachment, thereby damaging the attachment. The wall ensures that a force applied by the user to the stop via the endoscope does not have to be resisted entirely by the stop, as the wall is able to assist in providing a reaction force to counteract the force applied by the user, absorbing forces which would otherwise risk damaging the attachment.

In some embodiments, the attachment has a longitudinal portion extending between a distal end portion and a proximal end portion of the attachment, and the applicator comprises a distal end support for supporting the distal end portion of the attachment, and a proximal end support for supporting the proximal end portion of the attachment. As the attachment is supported at each end, sterilisation is facilitated. Furthermore, when the attachment is not supported along its longitudinal portion, the longitudinal portion is free from obstruction and able to move, for example as the endoscope is inserted.

In some embodiments, the applicator is reconfigurable to support an attachment of a different length by altering the distance between the distal end support and the proximal end support. This enables the applicator to be used in applying attachments of different sizes to correspondingly sized endoscopes, providing economies of scale by enabling at least a portion of the applicator to be made in the same dimensions and to be manufactured in the same way (e.g. using the same mould or moulds), regardless of the size of the attachment that it is intended to be used with.

In some embodiments, the distal end support is defined by an insert in the applicator. The use of an insert to define the distal end support enables the applicator to be easily modified to be used with an attachment of a different size by moving and/or replacing the insert. In some embodiments, the wall adjacent the stop is defined by the insert. This enables the insert to perform the two functions of supporting the distal end of the attachment as well as protecting the stop from damage. In some embodiments, the longitudinal length of the insert determines the distance between the distal and support and the proximal end support. This enables easy positioning of the insert in the applicator, for example by placing the insert to abut the distal end of the applicator.

In some embodiments, the attachment has a longitudinal portion with a gap extending between a distal end portion and a proximal end portion of the attachment, the gap being defined between two longitudinal edges of the attachment. The longitudinal portion is resiliently deformable from a rest configuration to allow insertion of the endoscope and to exert a gripping force on it. The gripping force exerted by the longitudinal portion of the attachment enables the attachment to be held firmly in place on the endoscope and therefore less liable to move relative to the endoscope both as the endoscope and attachment are withdrawn from the applicator and in subsequent use of the endoscope with the attachment. It therefore assists in enabling the attachment to move with the endoscope as the endoscope is withdrawn from the applicator. The gripping force ensures that the friction between the attachment and the endoscope is larger than the friction between the attachment and the applicator, thereby causing the attachment to move with the endoscope as the endoscope is withdrawn from the applicator. In embodiments in which the attachment is used to define a fluid conduit partially defined by the outer surface of the endoscope, the gripping force facilitates a seal between the attachment and the endoscope, which ensures that the fluid remains in the conduit and does not leak around the sides of the conduit between the endoscope and the attachment.

In some embodiments, the proximal end portion of the attachment defines a guiding surface for guiding the endoscope into the attachment. This facilitates ease of use.

In some embodiments, the applicator comprises an edge retainer for engaging the longitudinal edges. The edge retainer is arranged to limit torsion of the longitudinal portion of the attachment as the endoscope is inserted. The edge retainer is arranged in these embodiments to retain the edges by preventing or limiting their torsion or rotation about a longitudinal axis. The edge retainer thus protects the attachment from damage due to torsion while the endoscope is in the applicator, and while the endoscope is being inserted or withdrawn from the applicator.

In some embodiments, the attachment is arranged such that insertion of the endoscope into the attachment causes the edges to move to a position in which the longitudinal retainer is disposed relative to the edges to prevent or limit torsion or rotation of the attachment. The longitudinal portion of the attachment is resiliently deformable from a rest configuration to exert a gripping force on the endoscope, and the edge retainer is arranged to prevent or limit torsion or rotation of the attachment when the longitudinal portion is in its deformed position, i.e. when the endoscope is present. This allows the attachment to remain in its rest configuration while the device is stored before it is used to prevent pre-stressing. This ensures that the gripping force applied by the attachment to the endoscope is not reduced due to the attachment having experienced fatigue due to a long period of storage in a deformed configuration. The attachment is stored in its rest configuration and is only moved away from this configuration at the time when the endoscope is inserted. The edge retainer extends along the longitudinal portion to keep the edges in line when the endoscope is being inserted. This prevents the attachment from twisting as the endoscope is inserted, which could cause damage to the attachment. Specifically, in some embodiments, the edge retainer comprises a pair of rails.

The edge retainer may be holding the longitudinal edges further apart than in their rest configuration in some embodiments. In these embodiments, the edge retainer is used to hold the edges further apart than in their rest configuration to reduce the gripping force on the endoscope inside the attachment. The edge retainer is configured such that, while the endoscope is in the attachment inside the applicator, the edges are slightly further apart than they would have been if the edge retainer were not there. This reduces the gripping force applied by the attachment to the endoscope, thereby making it easier for the user to rotate the endoscope with respect to the attachment in order to align them correctly. The gripping force on the endoscope is nevertheless still strong enough to ensure that the attachment moves with the endoscope as the endoscope is withdrawn from the applicator. On withdrawal, the applicator deforms closer to its rest configuration, thereby increasing the gripping force on the endoscope.

In some embodiments, the applicator comprises a heating arrangement for heating the endoscope while the endoscope is in the applicator. It has been found that condensation is prone to appear on a lens of an endoscope during a period of time immediately after the endoscope is inserted into a patient for surgery. It can take up to several minutes for the condensation to clear. The condensation on the lens reduces the visibility through the lens. This can cause a time delay before the surgical procedure can begin, or increase the risk to the patient if the surgical procedure begins before the condensation has cleared.

One explanation of this condensation is that the condensation is caused by the rapid change in temperature and other environmental conditions experienced by the endoscope from room-temperature conditions (about 20° C.) before the endoscope is inserted into the patient to body-temperature conditions (about 37° C.) after the endoscope has been inserted into the patient.

It has been found that pre-heating the endoscope to human body temperature prior to inserting the endoscope into the patient substantially eliminates the appearance of any condensation on the lens after entry into the patient. It has further been found that the device as described above can be utilised in heating the endoscope, thereby solving the problem of how to prevent condensation on the lens at the beginning of surgery, while also providing the same advantages as those described above in addition to this. In these embodiments, there is therefore no requirement for additional equipment to be used in heating the endoscope.

A further aspect of the invention provides an applicator for applying an attachment to an endoscope. The attachment has a longitudinal portion extending between a distal end portion and a proximal end portion of the attachment. The applicator comprises a distal end support for supporting the distal end portion of the attachment, and a proximal end support for supporting the proximal end portion of the attachment. The applicator is reconfigurable to support an attachment of a different length by altering the distance between the distal end support and the proximal end support.

A further aspect of the invention relates to an applicator for applying an attachment to an endoscope. The attachment has a longitudinal portion extending between a distal end portion and a proximal end portion of the attachment. The applicator comprises a distal end support for supporting the distal end portion of the attachment, and a proximal end support for supporting the proximal end portion of the attachment. The applicator comprises a retainer extending longitudinally between the distal end support for limiting torsion of the longitudinal portion of the attachment.

A further aspect of the invention relates to an applicator for applying an attachment to an endoscope. The attachment has a longitudinal portion extending between a distal end portion and a proximal end portion of the attachment. The applicator comprises a distal end support for supporting the distal end portion of the attachment, and a proximal end support for supporting the proximal end portion of the attachment. The applicator comprises a heating arrangement for heating the endoscope while the endoscope is in the applicator. The arrangement may be as described above.

A further aspect of the invention relates to an endoscope heater comprising a heating element and a holder for holding an endoscope with the heating element longitudinally along the endoscope. The endoscope heater may be configured as the device or applicator described above, or otherwise. For example the holder may be the applicator described above, with the heater (e.g. in the form of a heating element) embedded in it. In other embodiments, the holder may be the attachment described above. In this case, the heater/heating element, e.g. a resistive wire, may be embedded in the attachment. In this last case, the heater is conveniently withdrawn from the applicator together with the endoscope so that the endoscope can be heated in situ during an operation.

A further aspect of the invention relates to a method of loading an attachment for an endoscope into an applicator. The attachment has a longitudinal portion extending between a distal end portion and a proximal end portion of the attachment. The applicator comprises a distal end support for supporting the distal end portion of the attachment, and a proximal end support for supporting the proximal end portion of the attachment. The method comprises inserting the attachment into the applicator; arranging the distal end portion of the attachment to be supported by the distal end support of the applicator; and arranging the proximal end portion of the attachment to be supported by the proximal end support of the applicator.

A further aspect of the invention relates to a method of applying an attachment to an endoscope. The attachment is initially supported within an applicator, and an end of the attachment is accessible at an end of the applicator. The method comprises inserting an endoscope into the attachment while the attachment remains supported within the applicator to apply the attachment to the endoscope; and withdrawing the endoscope from the applicator, wherein the applied attachment moves with the endoscope.

In some embodiments, the method further comprises the step of rotationally aligning the attachment and the endoscope while the attachment remains supported within the applicator. This enables alignment of the endoscope and the attachment to be achieved when the attachment is still being protected by the applicator, rather than afterwards, when the attachment is more prone to being damaged.

Embodiments of the invention are now described by way of example only and with reference to the accompanying drawings, in which:

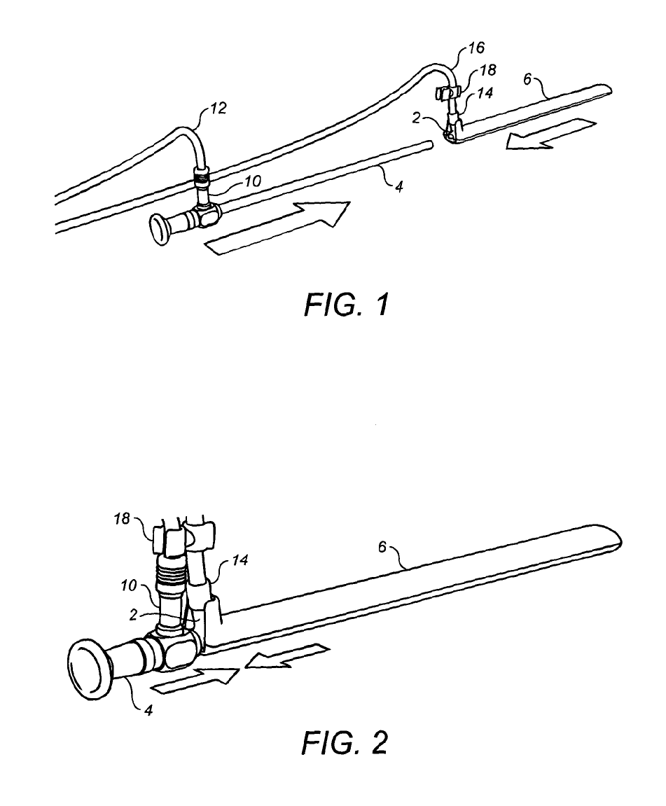

FIG. 1 depicts a perspective view of an endoscope ready to be inserted into an attachment contained within an applicator;

FIG. 2 depicts a perspective view of the endoscope inserted into the attachment within the applicator;

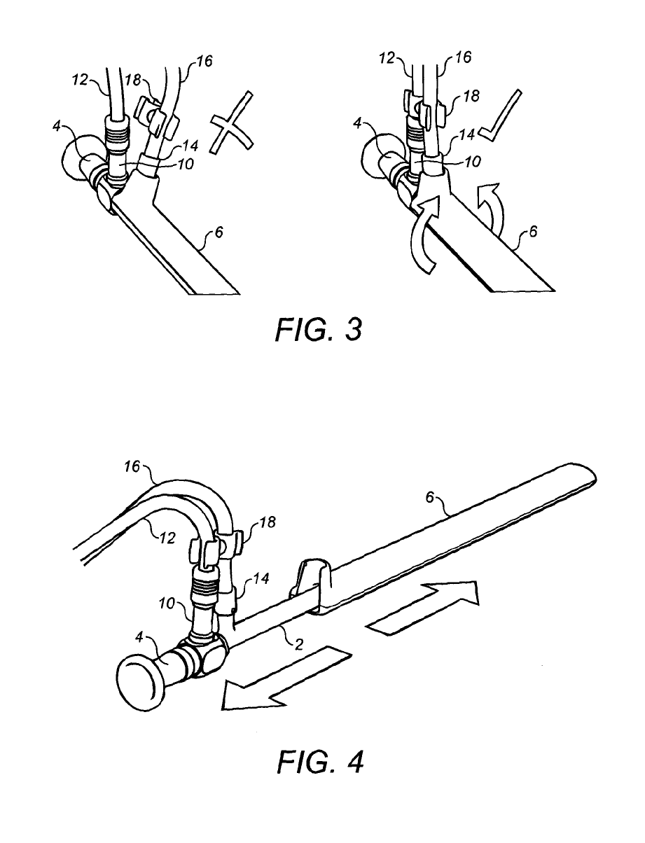

FIG. 3 illustrates the method of aligning the attachment and the applicator with respect to the endoscope;

FIG. 4 depicts a perspective view of the endoscope and the attachment being withdrawn together from the applicator;

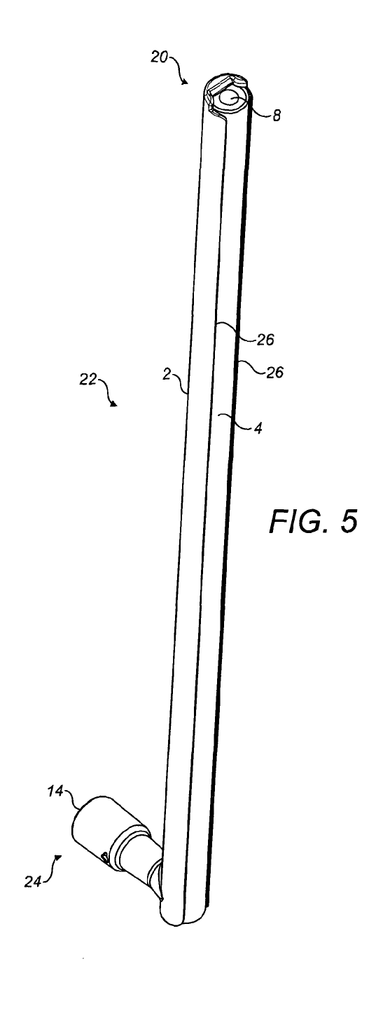

FIG. 5 depicts a perspective view of the endoscope and the attachment applied to the endoscope after they have been withdrawn from the applicator;

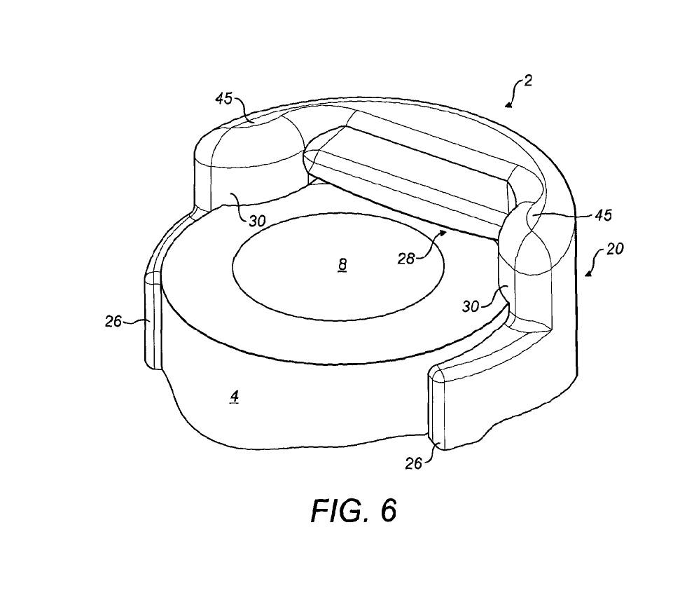

FIG. 6 depicts a perspective view of a distal end portion of the attachment and the endoscope according to FIG. 5;

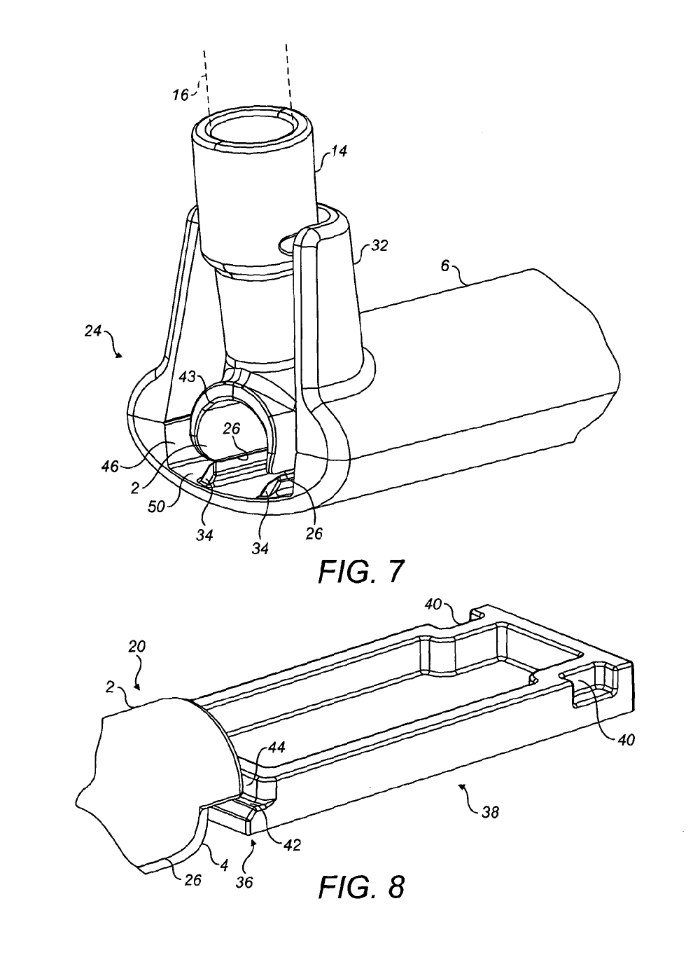

FIG. 7 depicts a perspective view of a proximal end portion of the attachment and the applicator while the attachment is contained within the applicator before the endoscope is inserted;

FIG. 8 depicts a perspective view of the attachment, the attachment being applied to the endoscope, and an insert contained within the applicator (the remainder of the applicator is not shown for clarity);

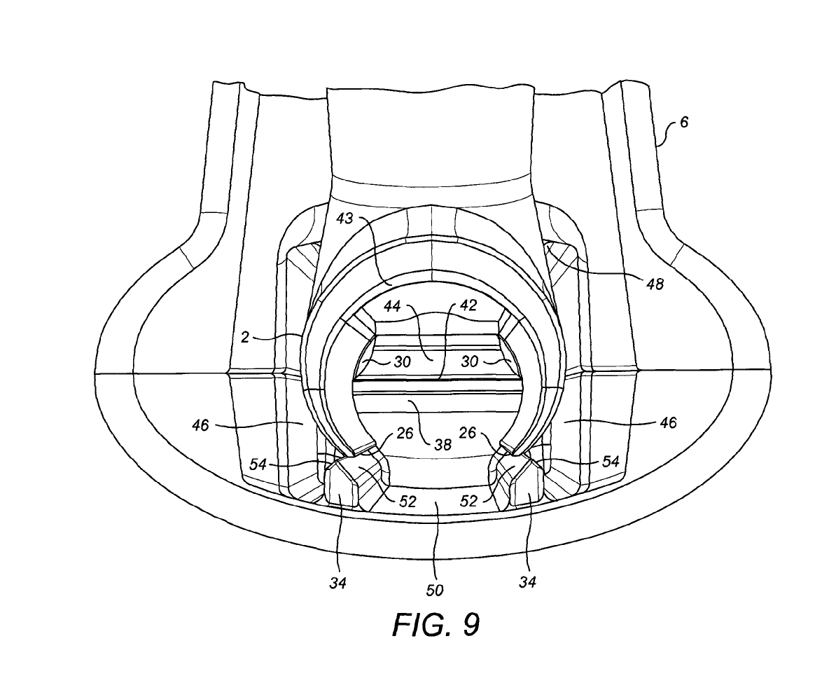

FIG. 9 depicts a rear view from the proximal end of the attachment and the applicator before the endoscope is inserted;

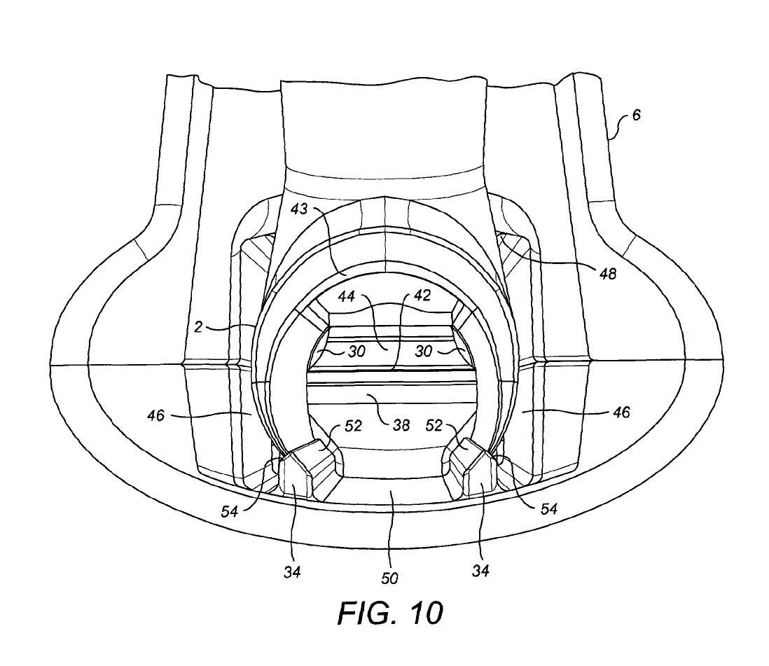

FIG. 10 depicts a view from the proximal end of the attachment and the applicator after the endoscope has been inserted (the endoscope not being shown for reasons of clarity);

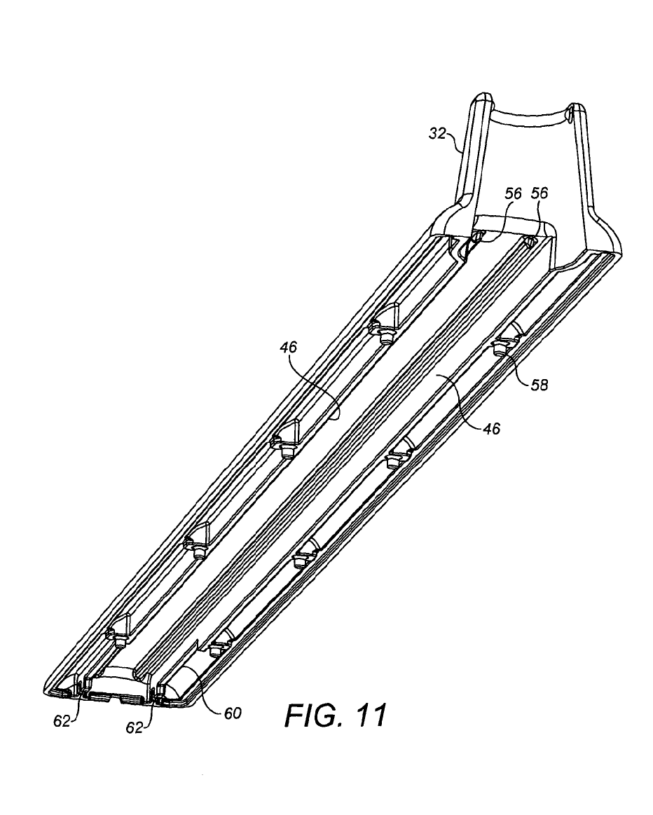

FIG. 11 depicts a portion of the applicator illustrating how the insert locates; and

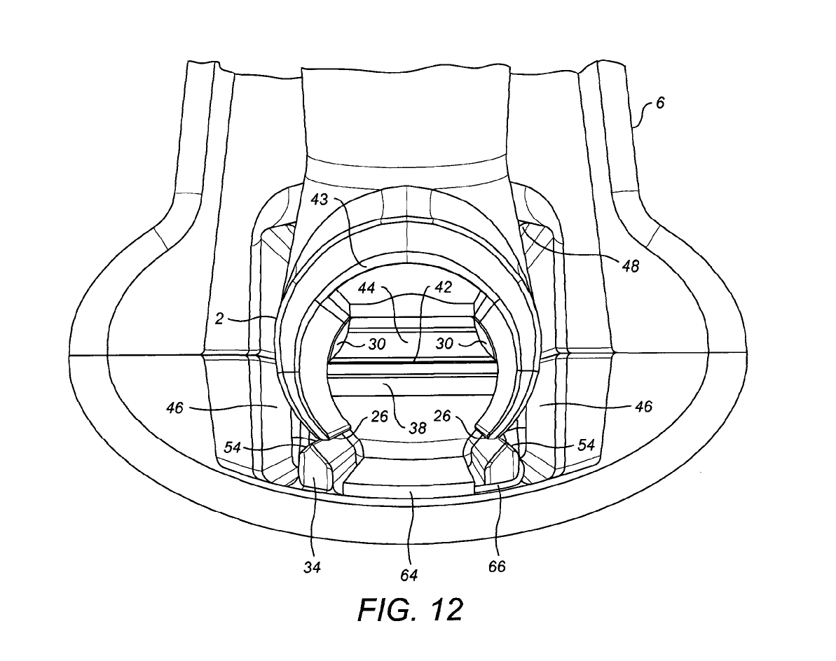

FIG. 12 depicts an endoscope heater.

With reference to FIGS. 1 to 4, by way of overview, a method of applying an attachment 2 to an endoscope 4 using an applicator 6 is described. The attachment 2 and applicator 6 are provided assembled together in a sterilised pack, e.g. a medical plastic pouch (Tyvek) or a foil pack. In use, the foil pack can be opened in the sterile environment of an operating theatre, dispensing with the need for further sterilisation. A user, e.g. scrub nurse, opens the pack and removes the applicator 6 ready for use.

FIG. 1 shows the endoscope 4 ready to be inserted into the attachment 2. The attachment 2 is contained within an applicator 6, which acts as a housing and surrounds the attachment 2. The endoscope 4 is a laparoscope and comprises a rigid cylindrical shaft and has a lens 8 at its distal end. A connector 10 for a fibre optic bundle 12 providing a light source is provided on the endoscope 4 close to its proximal end. The applicator 6 is made of a transparent material and is shaped to receive the endoscope 4 within the attachment 2 and to extend along substantially the entire length of the shaft of the endoscope 4. The attachment 2 also is arranged to extend along substantially the entire length of the shaft of the endoscope 4. The applicator 6 also extends substantially along the entire length of the attachment. A proximal end portion 24 of the attachment 2 comprises an inlet connector 14 for connecting to a supply hose 16 for fluid (such as carbon dioxide gas) to be transported to a distal end of the attachment 2.

The proximal end of the attachment 2 is exposed to enable the endoscope 4 to be inserted into it. The distal end of the endoscope 4 is introduced to the proximal end of the attachment 2, and the endoscope 4 is slid into the attachment 2 while the attachment 2 remains within the applicator 6. FIG. 2 shows the endoscope 4 after it has been fully inserted into the attachment 2. In this fully inserted position, it is possible for a user to align the endoscope 4 with respect to the attachment 2 by rotating the attachment 2 and applicator 6 with respect to the endoscope 4 about a longitudinal access. The connector 10 and the inlet connector 14 can therefore be aligned to extend in substantially the same direction. A clip 18 is provided to fasten the fibre optic bundle 12 to the supply hose 16, as illustrated in FIG. 3. The clip can be omitted or replaced by an alternative fastener, e.g. one or more cable ties. The endoscope 4 can then be removed from the applicator 6 together with the applied attachment 2, as shown in FIG. 4.

With reference to FIGS. 5 and 6, the endoscope 4 with the attachment 2 applied to it, removed from the applicator 6, is described. A distal end portion 20 of the attachment 2 is adjacent the distal end of the endoscope 4. A longitudinal portion 22 of the attachment 2 extends between the proximal end portion 24 and the distal end portion 20. The inlet connector 14 extends transversely away from a longitudinal portion of the proximal end portion 24. In a transverse cross-section, the longitudinal portion 22 is crescent or “C”-shaped and therefore partially encloses the endoscope 4. A gap is defined along the longitudinal portion 22 by two edges 26 extending in parallel in the longitudinal direction.

In use during surgery, the distal end portion 20 of the attachment 2 with the endoscope 4 is inserted through a cannula as described above. The proximal end portion 24 of the attachment 2 remains outside the patient.

At the distal end portion 20 of the attachment 2 (as shown in more detail in FIG. 6), an outlet 28 is defined. The outlet 28 is for passing a fluid stream across the surface of the lens 8 on the endoscope 4 to keep the lens 8 clean and free from biological particulate during an operation. A conduit (not shown) extends along the longitudinal portion 22 and connects the inlet connector 14 at the proximal end portion 24 to the outlet 28. The conduit is defined in a space between the attachment 2 and the endoscope 4 on the inside of the attachment 2 and the outside of the endoscope 4.

The distal end portion 20 of the attachment 2 comprises two stops 30. Each stop 30 has a surface arranged to abut a part of the distal end surface of the endoscope 4 when the endoscope 4 has been fully inserted into the attachment 2. The stops 30 are located so as not to significantly restrict the field of vision through the lens 8.

With reference to FIG. 7, the proximal end portion 24 of the attachment 2 is shown, with the attachment 2 being supported by the applicator 6 (before the endoscope 4 is inserted). The applicator 6 comprises a proximal end support 32, which projects transversely away from the main body of the applicator 6 at a proximal end of the applicator 6. The proximal end support 32 comprises a surface arranged to meet with a corresponding surface on the inlet connector 14 in order to support the inlet connector 14. The proximal end support 32 comprises two lugs at opposite sides of the inlet arranged to meet with two corresponding detents at each side of the inlet. The proximal end support 32 is arranged such that the attachment 2 is removable from the applicator 6 in the longitudinal direction without the proximal end support 32 having to be manually disconnected from the inlet prior to the removal of the attachment 2. The proximal end support 32 supports the weight of the attachment 2 and enables it to hang below the proximal end support 32 when the applicator 6 is arranged such that the longitudinal direction is horizontal and the proximal end support 32 extends upwardly. The edges 26 of the attachment 2 then hang above two retaining rails 34, which extend longitudinally along the applicator 6 in parallel and aligned with the edges 26. These are further described below with reference to FIGS. 9 and 10.

With reference to FIG. 8, at the distal end portion 20 of the attachment 2, when the attachment 2 is within the applicator 6, part of the distal end portion 20 of the attachment 2 is supported by a distal end support 36 on the applicator 6. This is also the case when the endoscope 4 has been inserted into the attachment 2, prior to their removal from the applicator 6. The distal end support 36 is defined by an insert 38 within the applicator 6, which is fixed in place relative to the main body of the applicator 6 before the attachment 2 is put into position, such that the insert 38 is unable to move relative to the main body of the attachment 2. A detent 40 on each side near a distal end of the insert 38 serves to locate the insert 38 in the applicator 6, as described below with reference to FIG. 11. The distal end support 36 is situated at a predetermined distance from the distal end of the insert, at the proximal end of the insert. The distance between the distal and proximal ends of the insert 38 therefore determines the distance between the two supports for the attachment 2—the distal end support 36 and the proximal end support 32. This distance is set according to the longitudinal length of the attachment 2, which is itself set to accommodate an endoscope 4 of a particular longitudinal length such that the distal end of the endoscope 4 is able to reach the distal end portion 20 of the attachment 2, the attachment 2 also extending along substantially the entire longitudinal length of the shaft of the endoscope 4.

The distal end support 36 comprises a ledge 42 on the insert 38 that is in a plane parallel to the longitudinal direction, and is also parallel to the plane defined by the two edges 26 of the attachment 2. When the applicator 6 is oriented with the longitudinal axis being horizontal and the inlet pointing upwards, a longitudinal surface of each of the two stops 30 rests on the ledge 42.

A wall 44 adjacent the ledge 42 has a surface that extends from a distal end of the ledge 42, the surface being substantially perpendicular to the ledge 42. The wall 44 is arranged to abut distal end surfaces 45 (see FIG. 6) of the stops 30 on the attachment 2 (i.e. the surfaces that face away from the endoscope 4). If the endoscope 4 is inserted into the attachment 2 with a large enough force, it is possible that the stops 30 could be made to buckle or break off the attachment 2. The wall 44 prevents this by being positioned to provide a reaction force, via the distal end surfaces 45, to a force applied from the endoscope 4 towards the stops 30.

The ledge 42 does not extend far enough in the proximal longitudinal direction to contact the endoscope 4, which ensures that the endoscope 4 is able to be inserted up to the stops 30 without the ledge 42 interfering such that the stops 30 define a well-defined insertion depth.

With reference to FIGS. 9 and 10, the attachment 2 can be seen situated within the applicator 6. The proximal end of the attachment 2 defines a guiding surface 43 at its proximal end for guiding the endoscope 4 into the attachment 2. The guiding surface 43 is angled with respect to the transverse plane such that the edges 26 do not extend as far in the proximal longitudinal direction as the rest of the proximal end surface of the attachment 2. This ensures that, as the endoscope 4 is inserted, it first contacts the part of the guiding surface 43 on the opposite side to the edges 26, and then as the endoscope 4 is inserted the endoscope 4 causes the edges 26 to separate from each other to allow room for the endoscope 4 to be inserted.

The longitudinal portion 22 of the attachment 2 is between two internal side walls 46 of the applicator 6 and is between an upper wall 48 and a lower wall 50 (from which the retaining rails 34 extend). The walls surround the longitudinal portion 22 circumferentially and extend along its entire length. The upper wall 48 comprises a pair of stabilising rails (not shown), which are parallel to and opposite the retaining rails 34 (although not necessarily the same distance apart). The stabilising rails assist the proximal end support 32 and distal end support 36 in stabilising the attachment 2, particularly as the endoscope 4 is inserted. The stabilising rails contact the attachment 2 at all times.

As the longitudinal portion 22 is made of a resilient material, it has a rest configuration in which the edges 26 are a certain distance apart and define a gap of a certain size between them. The cross-sectional area defined within the longitudinal portion 22 in its rest configuration is such that the endoscope 4 cannot be accommodated in the longitudinal portion 22 without the longitudinal portion 22 being deformed away from its rest configuration. As the longitudinal portion 22 will tend to return to its rest configuration, it will therefore exert a gripping force on the endoscope 4. There is sufficient space inside the applicator 6 for the longitudinal portion 22 to deform as required to accommodate the endoscope 4.

Each of the retaining rails 34 comprises a riding surface 52 and a retaining surface 54. Each riding surface 52 is on the inside of the retaining rail 34 (i.e. it faces the other retaining rail) and each retaining surface 54 is on the outside of the retaining rail. Each riding surface 52 is inclined at an angle of about 38° with respect to a plane defined by the retaining rails 34 and is arranged such that the respective edge 26 will ride up it as the endoscope 4 is inserted and the edges 26 are forced apart. Each retaining surface 54 is inclined at an angle of about 38° with respect to the plane defined by the retaining rails 34 and is arranged such that the respective edge 26 will adjacent it (for example in contact with minimal or no gripping force, or with a small gap between them) once the edge 26 has ridden over a peak between the riding surface 52 and the retaining surface 54.

In its rest configuration, the longitudinal portion 22 is configured as shown in FIG. 9, with its edges 26 separated from the retaining rails 34 by a small distance (although in other embodiments, the edges 26 rest against the riding surfaces 52). As the endoscope 4 is inserted into the attachment 2, a portion of each edge 26 longitudinally near to the distal end of the endoscope 4 rides up the respective riding surface 52 and rides over the peak to be positioned outside the retaining surface 54. Once the endoscope 4 has been fully inserted, the attachment 2 and applicator 6 are configured as shown in FIG. 10, with the rails 34 disposed in the gap between the edges 26 (the endoscope 4 not being shown for clarity). As the endoscope 4 is withdrawn, the edges 26 slide longitudinally adjacent and parallel to the retaining surfaces 54, thus preventing torsion or rotation of the attachment 2 during the withdrawal of the endoscope.

In some alternative embodiments, the edges 26 will grip the holding surfaces 54. The retaining rails 34 are configured to hold the edges 26 of the longitudinal portion 22 of the attachment 2 apart at a distance larger than in the rest configuration. Although the edges 26 grip the holding surfaces 54, a gripping force is also applied to the endoscope 4, although not as strong a force as is applied once the endoscope 4 and attachment 2 have been removed from the applicator 6. This makes it easier for the user to rotate the endoscope 4 with respect to the attachment 2 to align them as described with respect to FIG. 3. The gripping force on the endoscope 4 is still sufficient for the attachment 2 to move with the endoscope 4 as the endoscope 4 is withdrawn from the applicator 6. This occurs naturally as the endoscope 4 is withdrawn, as the frictional force between the attachment 2 and the endoscope 4 is larger than the frictional force between the attachment 2 and the applicator 6.

With reference to FIG. 11, depicting a portion for assembly into the applicator 6 which defines the proximal end support 32 and a portion of each side wall 46, as well as a pair of locating rails 56 for assisting in locating the attachment 2 as the endoscope 6 is inserted and lugs 58 for aiding assembly of the applicator 6, a recess 60 is provided in each side wall 46 adjacent a distal end of the applicator 6 for accommodating the insert 38. A pair of protrusions 62 in the recess 60 is arranged to engage the pair of respective detents 40 in the insert 38 to locate the distal end of the insert 38 adjacent to the distal end of the applicator 6, so that the longitudinal position of the ledge 42 is determined by the length of the insert 38. The insert 38 is replaceable by a second insert of a different longitudinal length, thereby enabling the applicator 6 to be used with an attachment of a different longitudinal length. The insert 38 and/or the attachment 2 are colour coded to enable the user to determine the type of attachment 2 that is in the applicator 6 (the applicator 6 being transparent).

The attachment 2 is made of Polyamide 11 (PA 11) (Nylon 11) and the applicator 6 is made of Poly(methyl methacrylate) (PMMA).

It is to be understood that the above description of specific embodiments of the invention is given by way of example only and is not intended to limit the scope of the invention. Many modifications of the described embodiments, some of which are now described, are envisaged and intended to be covered by the appended claims.

In some embodiments, the attachment 2, applicator 6 and insert 38 are made of any medical grade Class IV to VI polymeric material, e.g. Radel A; Polyethersulfone; Radel R; Polyphenylsulfone and related/modified polymers; Polyetheretherketone (PEEK); Polyether Ketone Ketone (PEKK); Polyphenylene; Valox (TM) resins, for example based on Polyethyleneterephthalate (PET) or polybutyleneterephthalate (PBT); Polyethyleneterephthalate (PET); Polybutyleneterephthalate (PBT); Lexan Polycarbonates; Acrylonitrile Butadiene Styrene (ABS); Polypropylene; Polyimides; and Polyacrylates and/or blends of these polymers. Each part can be made of a different one of these materials or the same material can be used for all parts. The attachment 2, the applicator 6 and the insert 38 are manufactured using injection moulding techniques and then assembled after moulding.

In some embodiments, now described with reference to FIG. 12, the applicator comprises a heating arrangement for heating the endoscope while the endoscope is in the applicator. The heating arrangement comprises a resistive element 64 between the two rails 34, such that it can heat the endoscope through the gap between the edges 26 of the attachment 4 when the endoscope 2 is in the applicator 6. The resistive element 64 is disposed along the length of the applicator 6, extending over substantially all of its longitudinal extent, the region occupied by the endoscope, or only a portion of the longitudinal extent. Likewise, the resistive element 64 can be disposed in alternative configurations, for example at other locations, such as other locations inside the applicator 6 or around the applicator 6, for example wrapped around the applicator 6. A current return path is provided by a conductor 66 on the outside of one of the two rails. The resistive element 64 is connectable to a source of power external to the applicator via a connector at the distal end of the applicator 6 (not visible in FIG. 12). Alternatively, an onboard power source, such as a battery may be carried by the applicator 6, for example at the distal end.

In some alternative embodiments, the heating arrangement comprises an LED or a hot water conduit instead of the resistive element 64. In some embodiments, the heating arrangement (for example an LED) is located at the distal end of the applicator 6. In yet some further embodiments, the heating arrangement comprises its own source of energy that can be activated to heat the endoscope at the appropriate time, for example by a chemical reaction, e.g. triggered by insertion of the endoscope. In some embodiments, heating is triggered by the insertion of the endoscope 3 into the applicator 6, for example by triggering a switch disposed in the applicator 6 such that it is actuated by insertion of the endoscope 2, or by completing an electrical circuit with a conductive portion of the endoscope.

In some embodiments, the resistive element 64 (or another type of heating element, such as those described above) is embedded in the material forming the applicator 6 to provide an embedded heating element. In some embodiments where the applicator 6 is manufactured by moulding, for example, this is done by placing a resistive element such as a conductive mesh in the mould, for example an injection mould, used to mould the applicator 6 (or a part of the applicator 6). The embedded heating element is disposed between the rails 34 in some embodiments. Specifically, the embedded heating element may be placed in the applicator material between the rails 34. In some embodiments, a heating element can be embedded in other locations, additionally or alternatively, for example in the material forming an outer wall of the applicator.

In some embodiments, rather than to provide the heating element in the applicator 6, it is provided in the attachment 2, for example by embedding a resistive wire or other heating element in the material of the attachment 2 as described above for the applicator 6. The heating element may be embedded over substantially all of the circumferential and/or longitudinal extend of the attachment 2, or only a portion thereof. The heating element/resistive wire is connected to a connector at the proximal end of the attachment 2 for connection to a power source and/or control unit for heating the heating element/resistive wire. An embedded heater is described in WO2004/107815, incorporated herein by reference.

In some embodiments, there is no insert, but the distal end support and the wall are instead defined by the main body of the applicator. In yet other embodiments, the applicator is configurable by placing the insert in different fixing locations to vary the longitudinal distance between the proximal end support and the distal end support, rather than inter-changing differently sized inserts.

In some embodiments, the applicator does not or not fully circumferentially surround the attachment, although it still locates the distal end portion and the proximal end portion of the attachment relative to one another. In some embodiments, the applicator does not have any retaining rails along the lower wall and there may be no lower wall at all. Some embodiments omit the stabilising retaining rails along the upper wall. As long as the distal end support and the proximal end support are located relative to each other, any one or more of the walls can be omitted.

In some embodiments, the longitudinal portion of the attachment is configured to fully surround the endoscope, rather than leaving a gap along its longitudinal length. The attachment is still able to move with the endoscope as the endoscope is withdrawn from the applicator due to a larger frictional force between the attachment and the endoscope than the frictional force between the attachment and the applicator. In some embodiments, the edges along the longitudinal portion of the attachment are arranged to grip the retaining surfaces on the retaining rails before the endoscope is inserted, such that the endoscope can be inserted with reduced movement of the longitudinal portion of the attachment being required.

In some embodiments the conduit along the longitudinal portion is for access for instrumentation rather than for acting as a fluid conduit. The inlet of the embodiment described above is therefore replaced by an appropriate feature for the instrumentation conduit. In some embodiments, more than one conduit is provided, for example one fluid conduit and one instrumentation conduit. In some embodiments, the attachment does not extend along substantially the entire longitudinal length of the shaft of the endoscope, but only extends far enough such that in use it is not inserted in its entirety through the cannula, so as to provide, in use, access to the conduit from outside the body to transport fluid (or e.g. an instrument) to the distal end of the endoscope inside the body.

In some embodiments, there is only one stop on the attachment, rather than two. In some embodiments, the stop (or stops) is defined at a different location on the attachment than for the embodiment described above and is arranged to abut a different part of the endoscope, for example part of the proximal end portion of the endoscope.

In some embodiments, instead of the clip described above, another fastener is provided that applies a force which pulls the endoscope and the attachment together, which helps to ensure that the endoscope remains fully inserted into the attachment. In at least these embodiments, it is not necessary for the frictional force between the attachment and the endoscope to be larger than the frictional force between the attachment and the applicator when the endoscope is withdrawn from the applicator.

A wide range of materials and manufacturing techniques can be employed in addition to or instead of the ones described above. In particular the applicator is opaque or translucent, rather than transparent, in some embodiments.

Finally, while the above description has been made in terms of a laparoscope, the attachment and applicator are equally applicable to other kinds of endoscopes. In particular, in some embodiments, the endoscope is a flexible or semi-rigid endoscope and the attachment is similarly flexible.

1. A device for applying an attachment to an endoscope, the device comprising an applicator supporting the attachment, an end of the attachment being accessible such that the endoscope is insertable into the attachment in a longitudinal direction, the attachment being removably secured to the applicator to enable the attachment to be withdrawn from the applicator together with the endoscope.

2. A device as claimed in claim 1, wherein the attachment comprises a stop having a surface for contacting the endoscope to define a predetermined maximum insertion distance of the endoscope into the attachment.

3. A device as claimed in claim 2, wherein the surface is arranged to abut a portion of a distal end of the endoscope.

4. A device as claimed in claim 2, wherein the applicator comprises a wall adjacent the stop for exerting a reaction force to a longitudinal force applied to the surface.

5. A device as claimed in claim 1, wherein the attachment has a longitudinal portion extending between a distal end portion and a proximal end portion of the attachment, and the applicator comprises a distal end support for supporting the distal end portion of the attachment, and a proximal end support for supporting the proximal end portion of the attachment.

6. A device as claimed in claim 5, wherein the applicator is reconfigurable to support an attachment of a different length by altering the distance between the distal end support and the proximal end support.

7. A device as claimed in claim 5, wherein the distal end support is defined by an insert in the applicator.

8. A device as claimed in claim 7, wherein the attachment comprises a stop having a surface for contacting the endoscope to define a predetermined maximum insertion distance of the endoscope into the attachment, the insert defining a wall adjacent the stop for exerting a reaction force to a longitudinal force applied to the surface.

9. A device as claimed in claim 7, wherein the longitudinal length of the insert determines the distance between the distal end support and the proximal end support.

10. A device as claimed in claim 5, wherein the proximal end support is arranged to support the proximal end portion of the attachment at a projection extending away from the longitudinal portion.

11. A device as claimed in claim 10, wherein the proximal end support comprises a surface adapted to mate with a corresponding surface on the projection to removably secure the attachment to the applicator.

12. A device as claimed in claim 10, and a fastener for fastening the attachment to the endoscope.

13. A device as claimed in claim 12, wherein the fastener is arranged to fasten a hose connected to the proximal end portion of the attachment to a light conduit connected to the proximal end portion of the endoscope.

14. A device as claimed in claim 1, wherein the attachment has a longitudinal portion with a gap extending between a distal end portion and a proximal end portion of the attachment, the gap being defined between two longitudinal edges of the attachment.

15. A device as claimed in claim 14, wherein the longitudinal portion is resiliently deformable from a rest configuration to exert a gripping force on the endoscope.

16. A device as claimed in claim 15, wherein the proximal end portion of the attachment defines a guiding surface for guiding the endoscope into the attachment.

17. A device as claimed in claim 15, wherein the applicator comprises an edge retainer for limiting torsion of the longitudinal portion of the attachment.

18. A device as claimed in claim 17, wherein the attachment is arranged such that insertion of the endoscope into the attachment causes the longitudinal edges of the attachment to move to a position in which the longitudinal retainer limits torsion of the longitudinal portion of the attachment.

19. A device as claimed in claim 17, wherein the edge retainer is arranged such that it limits torsion of the longitudinal portion as the attachment is withdrawn from the applicator together with the endoscope.

20. A device as claimed in claim 19, wherein the edge retainer comprises two rails.

21. A device as claimed in claim 1, wherein the applicator provides a housing for the attachment, and the attachment is supported in the housing.

22. A device as claimed in claim 21, wherein the housing circumferentially surrounds the attachment.

23. A device as claimed in claim 1, wherein the applicator comprises a heating arrangement for heating the endoscope while the endoscope is in the applicator.

24. A device as claimed in claim 23, wherein the heating arrangement is arranged to heat a portion of the endoscope through a longitudinal gap in the attachment.

25. A device as claimed in claim 23, wherein the applicator comprises two rails for limiting torsion of the longitudinal portion of the attachment, the heating arrangement being disposed between the two rails.

26. A device as claimed in claim 23, the heating element comprising a resistive element disposed along the length of the device.

27. A device as claimed in claim 23, wherein the heating arrangement is arranged to heat the portion of the endoscope that is in the applicator substantially along its entire length.

28. A device as claimed in claim 23, wherein the heating arrangement is arranged to heat a distal end of the endoscope.

29. An applicator for applying an attachment to an endoscope, the attachment having a longitudinal portion extending between a distal end portion and a proximal end portion of the attachment, the applicator comprising a distal end support for supporting the distal end portion of the attachment, and a proximal end support for supporting the proximal end portion of the attachment, wherein the applicator is reconfigurable to support an attachment of a different length by altering the distance between the distal end support and the proximal end support.

30. An applicator as claimed in claim 29, wherein the distal end support is defined by an insert in the applicator.

31. An applicator as claimed in claim 30, wherein the distal end support comprises a ledge on the insert for supporting the attachment.

32. An applicator as claimed in claim 31, wherein the insert defines a wall for abutting the distal end portion of the attachment, the wall being substantially perpendicular to the ledge.

33. An applicator as claimed in claim 30, wherein the longitudinal length of the insert determines the distance between the distal end support and the proximal end support.

34. An applicator as claimed in claim 29, wherein the proximal end support is arranged to support the proximal end portion of the attachment at a projection extending away from the longitudinal portion.

35. An applicator as claimed in claim 34, wherein the proximal end support comprises a surface adapted to mate with a corresponding surface on the projection to removably secure the attachment to the applicator.

36. An applicator as claimed in claim 29, wherein the applicator provides a housing for the attachment.

37. An applicator as claimed in claim 36, wherein the housing is arranged to circumferentially surround the attachment.

38. An applicator as claimed in claim 29, wherein the applicator comprises a heating arrangement for heating the endoscope while the endoscope is in the applicator.

39. An applicator for applying an attachment to an endoscope, the attachment having a longitudinal portion extending between a distal end portion and a proximal end portion of the attachment, the applicator comprising a distal end support for supporting the distal end portion of the attachment, and a proximal end support for supporting the proximal end portion of the attachment, wherein the applicator comprises a retainer extending longitudinally between the distal end support and the proximal end support for locating in a longitudinal gap of the attachment to limit torsion of the longitudinal portion of the attachment.

40. An applicator as claimed in claim 39, wherein the retainer comprises two rails for engaging respective sides of the longitudinal gap.

41. An applicator as claimed in claim 40, wherein the rails are substantially parallel.

42. An applicator for applying an attachment to an endoscope, the attachment having a longitudinal portion extending between a distal end portion and a proximal end portion of the attachment, the applicator comprising a distal end support for supporting the distal end portion of the attachment, and a proximal end support for supporting the proximal end portion of the attachment, wherein the applicator comprises a heating arrangement for heating the endoscope while the endoscope is in the applicator.

43. An applicator as claimed in claim 42, wherein the heating arrangement is arranged to heat a portion of the endoscope through a longitudinal gap in the attachment.

44. An applicator as claimed in claim 42, wherein the applicator comprises two rails for limiting torsion of the longitudinal portion of the attachment, the heating arrangement being disposed between the two rails.

45. An applicator as claimed in claim 42, the heating element comprising a resistive element disposed along the length of the applicator.

46. An applicator as claimed in claim 42, wherein the heating arrangement is arranged to heat the portion of the endoscope that is in the applicator substantially along its entire length.

47. An applicator as claimed in claim 42, wherein the heating arrangement is arranged to heat a distal end of the endoscope.

48. An endoscope heater comprising a heating element and a holder for holding an endoscope with the heating element longitudinally along the endoscope.

49. A method of loading an attachment for an endoscope into an applicator, wherein the attachment has a longitudinal portion extending between a distal end portion and a proximal end portion of the attachment, and the applicator comprises a distal end support for supporting the distal end portion of the attachment, and a proximal end support for supporting the proximal end portion of the attachment, the method comprising:
inserting the attachment into the applicator;
arranging the distal end portion of the attachment to be supported by the distal end support of the applicator; and
arranging the proximal end portion of the attachment to be supported by the proximal end support of the applicator.

50. A method as claimed in claim 49, wherein the distal end support is defined by an insert, and the method comprises the step of arranging the insert within the applicator prior to inserting the attachment into the applicator.

51. A method of applying an attachment to an endoscope, the attachment initially being supported within an applicator, an end of the attachment being accessible at an end of the applicator, the method comprising:
inserting an endoscope into the attachment while the attachment remains supported within the applicator to apply the attachment to the endoscope; and
withdrawing the endoscope from the applicator, wherein the applied attachment moves with the endoscope.

52. A method as claimed in claim 51, further comprising the step of rotationally aligning the attachment and the endoscope while the attachment remains supported within the applicator.

53. A method as claimed in claim 52, further comprising the step of fastening the attachment to the endoscope using a fastener after they have been aligned.

54. A method as claimed in claim 51, further comprising the step of heating the endoscope while the endoscope is in the applicator.
